(19) United States
(12) Patent Application Publication
(10) Pub. No.: US 2011/0117541 A1
(43) Pub. Date: May 19, 2011

(54) Method for Screening of Agents for the Prevention of Hepatitis C Virus Infection with Cell Culture Tool

(76) Inventor: Albert P. Li, Columbia, MD (US)

(21) Appl. No.: 12/962,554

(22) Filed: Dec. 7, 2010

Related U.S. Application Data
(63) Continuation-in-part of application No. 11/668,155, filed on Jan. 29, 2007, now abandoned.

(51) Int. Cl. C12Q 1/70

(52) U.S. Cl. 435/5

(57) ABSTRACT

The invention relates to an improved method of screening of anti-HCV agents that may have an efficacy for prevention of hepatitis C virus. The method involves the isolation and cryopreservation of HCV-infected hepatocytes from multiple infected individuals. The isolated and cryopreserved hepatocytes are stored in a cryopreservation bank made up of HCV-infected hepatocytes representing the different genotypes of HCV. These stored hepatocytes then are co-cultured in a culture medium with uninfected hepatocytes, and anti-HCV screening of the hepatocytes is done by subjecting HCV infected hepatocytes and uninfected hepatocytes in parallel to the actions of different anti-HCV compounds at various concentrations. An effective anti-HCV agent will lead to prevention of increase in concentration of HCV content of uninfected cells in the co-culture.

### CROSS REFERENCE TO RELATED APPLICATIONS

This application is a continuation-in-part of U.S. patent application Ser. No. 11/668,155 filed on Jan. 29, 2007, and claims priority from U.S. Provisional Application No. 60/518331 filed Nov. 10, 2003, which are incorporated herein by reference.

### FIELD OF THE INVENTION

The field of the invention generally relates to a novel method for the selection of drug candidates for the prevention of hepatitis C virus infection by use of a novel cell culture tool.

### BACKGROUND OF THE INVENTION

The hepatitis C virus or HCV, first identified in 1989, is the major agent of the viral infections that once was termed non-A non-B hepatitis. The term “non-A non-B” was introduced in the 1970s to describe hepatitis of which the etiological agents, not yet identified, appear serologically different from hepatitis A and B based on immunological tests. HCV infection is often fatal and has been reported to infect 170 million individuals worldwide. Interferon and ribavirin are only moderately effective in the control of the progress, but not the cure, and are associated with myriad undesirable side effects.

A major problem with the discovery and development of anti-HCV drugs is the absence of an effective experimental system for the evaluation of pharmacological effects. The general approach is to screen for the inhibition of the expression of HCV genes using cell lines transfected with portions of the HCV genome. This screening assay has limited use as neither the HCV genome nor the cell lines are representative of the situation in vivo.

The confirmatory test for the efficacy of anti-HCV drug candidates is performed in nonhuman primates, with chimpanzee as the only acceptable animal model. The use of chimpanzees is expensive, requires a high quantity of the test materials, and is often considered to be inhumane.

A further complication towards treatment is the multiple genotypes of HCV. The most commonly used classification of Hepatitis C virus has HCV divided into the following genotypes: 1, 2, 3, 4, 5, 6, 7, 8, 9, 10 and 11. The HCV genotypes are broken down into sub-types, some of which include: 1a, 1b, 1c; 2a, 2b, 2c; 3a, 3b; 4a, 4b, 4c, 4d, 4e; 5a; 6a; 7a, 7b; 8a, 8b; 9a; 10a; and 11a. It is believed that the hepatitis C virus has evolved over a period of several thousand years to result in the current general global patterns of genotypes and subtypes, as listed below:

1a—mostly found in North and South America; also common in Australia;

1b—mostly found in Europe and Asia;

2a—is the most common genotype 2 in Japan and China;

2b—is the most common genotype 2 in the U.S. and Northern Europe;

2c—the most common genotype 2 in Western and Southern Europe;

3a—highly prevalent here in Australia (40% of cases) and South Asia;

4a—highly prevalent in Egypt;

4c—highly prevalent in Central Africa;

5a—highly prevalent only in South Africa;

6a—restricted to Hong Kong, Macau and Vietnam;

7a and 7b—common in Thailand;

8a, 8b and 9a—prevalent in Vietnam;

10a and 11a—found in Indonesia;

In North America, genotype 1a predominates, followed by 1b, 2a, 2b, and 3a. In Europe, genotype 1b is predominant, followed by 2a, 2b, 2c and 3a. Genotypes 4 and 5 are found almost exclusively in Africa. The discovery of anti-HCV drugs is complicated by that HCV of different genotypes are known to have different responsiveness to treatment. For instance, genotypes 1 and 4 are less responsive to interferon-based treatment than genotypes 2, 3, 5 and 6. An ideal screening assay for the discovery of anti-HCV agents would allow the evaluation of the agents towards HCV of multiple genotypes.

Ito et al teaches that hepatocytes cultured from HCV patients continue to support HCV replication. See Ito et al. in Cultivation of hepatitis C virus in primary hepatocyte culture from patients with chronic hepatitis C results in release of high titre infectious virus. Journal of General Virology (1996), 77, 1043-1054.

Li teaches that cryopreserved hepatocytes can be cultured as monolayer cultures. See Li in Human hepatocytes: Isolation, cryopreservation and applications in drug development. Chemico-Biological Interactions 168 (2007) 16-29.

The inventor believes that there is a need for an effective screen for anti-HCV compounds that is representative of the situation in vivo as well as allowing the evaluation of compounds that prevent infection of HCV from multiple genotypes of HCV.

### SUMMARY

A method for the screening of anti-HCV drug candidates for preventing HCV is described. In part, the novelty of the method is the banking of cryopreserved hepatocytes infected by different HCV genotypes isolated from livers of HCV-infected patients, culturing of such hepatocytes in multi-well plates, and screening for anti-HCV drug candidates for effectiveness towards inhibition of HCV transmission from infected hepatocytes to uninfected hepatocytes. The novelty and advantages of the method over the current art may include one or more of the following:

1. Banking of cryopreserved HCV-infected hepatocytes that consists of multiple genotypes. The collection of hepatocytes from different patients infected by the multiple genotypes of HCV allows evaluation of anti-HCV compounds towards HCV of multiple genotypes.

2. Hepatocytes derived from HCV patients represent the actual infected cells in humans, and thereby would not have the potential artifacts of engineered cell lines or hepatocytes infected with HCV after culturing.

3. Culturing of hepatocytes from the cryopreserved HCV-hepatocyte bank for anti-HCV screening provides a supply of cryopreserved hepatocytes. The use of cryopreserved hepatocytes allows the cells to be fully characterized (e.g. genotyping of the HCV; rate of HCV replication). Screening for anti-HCV agents can be performed using the most appropriate cells. One of the more important advantages is that one can perform the screening using multiple lots of hepatocytes, with each lot representing cells infected by HCV of a specific genotype.

The inventor believes that this novel method can significantly enhance the efficiency of discovery of anti-HCV drugs that prevent the transmission of HCV.

In one general aspect there is provided a method for co-culturing HCV-infected and uninfected human hepatocytes to screen for agents that prevent the transmission of HCV. The method includes the steps of:

retrieving HCV infected and uninfected hepatocyte cells from a cryopreservation bank;

thawing the cells in a warm water bath;

suspending the cells in a medium;

culturing the cells in a plate with multiple inner wells;

interconnecting the inner wells with a fluid medium;

providing one or more anti HCV agents; and

quantifying the HCV content of the co-culture by quantification of HCV RNA.

Embodiments of the method may include one or more of the following features. For example, the anti-HCV agent may be added to the fluid medium used for interconnecting the wells. Multiple anti-HCV agents may be used. Multiple anti-HCV agents may be added at different concentrations.

The co-cultured uninfected hepatocytes and infected hepatocytes may be connected by a fluid medium. The fluid medium used for interconnecting the wells may be DMEM/F12 medium containing 10% of fetal calf serum (FCS), insulin (10 ug/mL), and dexamethasone (100 nM).

The quantification of HCV RNA may be performed by RT-PCR. The temperature of the warm water bath may be approximately 37° C. The suspending medium may be DMEM/F12 medium containing 10% of fetal calf serum (FCS), insulin (10 ug/mL), and dexamethasone (100 nM).

The cell plate may be a collagen-coated plate. The cell plate may have six inner wells. Three wells may be used for culturing infected hepatocytes. Three wells may be used for culturing uninfected hepatocytes.

Different genotypes of HCV infected cells may be co-cultured with uninfected hepatocytes in multiple cell plates.

In another general aspect, there is provided a method for co-culturing infected and uninfected hepatocytes in a medium to evaluate a level of HCV infection through quantification of HCV production by infected hepatocytes. The method includes: extraction of total RNA of uninfected hepatocytes;

quantification of RNA of uninfected hepatocytes; and

quantification of an HCV titer of the medium and infected hepatocytes by quantification of RNA of HCV.

Embodiments of the method may include one or more of the feature described above or the following. For example, the quantification of RNA of HCV may be performed by RT-PCR.

In another general aspect, a cell culture tool includes a body, an outer wall extending from the body, and more than one vessel defined by the configuration of the body. Each vessel has a top edge below a rim of the outer wall.

Implementation may include one or more of the following features. For example, the body may have a flat surface with each vessel comprising a depression in the flat surface of the body, the depression configured to contain a volume of fluid. The vessel may have a cylindrical wall and a circular bottom and the outer surface of the body may be in the shape of a rectangular plate. The height of the outer wall may be about 20 millimeters.

In one implementation, each vessel comprises a cup connected to the body, each cup having a top edge below the rim of the outer wall. In another implementation, the vessel includes a container having a container wall with a top edge, the height of the container wall being about 4 millimeters. In a further implementation, each vessel comprises a partition wall dividing the space defined within the perimeter of the outer wall, the partition wall having a top edge.

In another general aspect, a multi-well culture dish includes a base having a flat surface with a plurality of wells and an outer wall surrounding the base. Each of the wells includes a containing wall with a height lower than the height of the outer wall. Implementation may include one or more of the features described above and the dish may also include six wells.

In another general aspect, multiple culture vessels can be connected using tubings, with or without a device (e.g. a pump) to circulate the fluid.

In another general aspect, a method of interacting a substance with more than one type of cell material in a culture dish having a plurality of wells includes depositing a different type of the cell material in separate wells of the culture dish, interconnecting the wells with a fluid medium, and adding the substance to the fluid medium. In various implementations, the substance may include a chemical or a drug.

In another general aspect, a method of metabolizing a drug in a multi-well culture dish includes depositing different types of cell material in separate wells of the multi-well culture dish, connecting the separate wells with a fluid media, and introducing the drug into the fluid media.

Implementation may include one or more of the following features or any of the features described above. For example, the cell material may include liver, kidney, spleen or lung cells, any cells that can be cultured, and/or tissue fragments or fractions.

In another general aspect, a method of metabolizing a drug in a cell culture dish having a body with six wells and a wall surrounding the six wells includes depositing kidney cells in a first of the six wells, liver cells in a second of the six wells, heart cells in a third of the six wells, lung cells in a fourth of the six wells, spleen cells in a fifth of the six wells, and brain cells in a sixth of the six wells, filling the dish with a fluid medium to fluidly interconnect the six wells, and introducing the drug into the fluid medium.

In another general aspect, a method of co-culturing different cells in individual wells includes overfilling each well to fluidly interconnect the wells so the different cells in the individual wells communicate through a common fluid medium.

The method may include various implementations. For example, the different cells in the individual wells comprise liver cells in a first well, kidney cells in a second well, heart cells in a third well, spleen cells in a fourth well, brain cells in a fifth well, and lung cells in a sixth well. In another implementation, the different cells in the individual wells comprise liver cells in a first, second and third well and heart cells in a fourth, fifth, and sixth well. In a further implementation, the method includes introducing a substance into the common fluid medium so that the different cells in the individual wells are in contact with the same substance.

In another general aspect, a method of testing the safety and efficacy of a drug in a culture dish having separate wells includes depositing different cells of an organism in the separate wells of the culture dish, depositing a harmful agent in another of the separate wells, interconnecting the separate wells with a fluid medium, and introducing a dose of the drug into the fluid medium.

The method may include one or more of the following features or any of the features described above. For example, the method may include determining whether the different cells of the organism are harmed by the dose of the drug, determining whether the harmful agent is diminished by the dose of the drug, and/or increasing the dose of the drug if the different cells of the organism are not harmed and the harmful agent is not diminished.

The harmful agent may include tumor cells and the drug may include an anti-tumor medication. The different cells of the organism may include liver, kidney, heart, lung, spleen, and/or brain cells of the human body.

The method may further include increasing the dose of the drug until the drug harms the different cells of the organism and designating the dose of the drug at which the different cells of the organism are harmed as a toxic dose level. The method also may include increasing the dose of the drug until the effect of the harmful agent is reduced and designating the dose of the drug at which the effect of the harmful agent is reduced as an effective dose level.

The harmful agent may be cholesterol, the drug may be an anti-cholesterol drug, and the different cells may include liver cells. In another implementation, the harmful agent includes cancer cells and the drug is an anti-cancer medication that has an undesirable toxicity above a certain dose.

In another general aspect, a method of co-culturing cells in a multi-well dish includes culturing a first cell type in a first well of the multi-well dish and culturing a second cell type in a second well of the multi-well dish. The cells cultured in the second well may provide metabolites that benefit the growth of the first cell type.

In another general aspect, a method of evaluating whether a first cell type can enhance the growth of a second cell type includes culturing the first cell type in a first well, culturing the second cell type in a second well, fluidly interconnecting the first well and the second well, and examining the impact of the cultured first cell type on the growth of the second cell type.

The cell culture tool provides a convenient way for multiple cell types to be co-cultured but yet physically separate so that the individual cell types can be evaluated separately after co-culturing in the absence of the co-cultured cells.

The tool allows the culturing of cells in individual wells under different conditions, such as, for example, different attachment substrate, different media, or different cell types, followed by allowing the different wells to intercommunicate via a common medium. After culturing as an integrated culture with a common medium, the medium can be removed, and each well can be subjected to independent, specific manipulations, such as, for example, lysis with detergent for the measurement of specific biochemicals or fixation and staining for morphological evaluation.

As described above in the method, an application is the culturing of multiple primary cells from different organs (e.g. liver, heart, kidney, spleen, neurons, blood vessel lining cells, thyroidal cells, adrenal cells, iris cells, cancer cells) so the plate, after the establishment of individual cell types and flooding, represents an in vitro experimental model of a whole animal. Another application of the culture tool is to evaluate the effect of a substance on multiple cell types. In drug discovery and development, this culture system can be used to evaluate metabolism of a new drug or drug candidate by cells from multiple organs or the effect of a drug or drug candidate on the function and viability of cells from multiple organs. An example of this application is to culture cells from multiple organs along with tumor cells, followed by treatment of the co-culture with an anticancer agent to evaluate toxicity of the agent to the cells of the different organs in comparison with its toxicity towards the cancer cells to evaluate the therapeutic index of the agent. In other words, each plate simulates the treatment of a whole animal with the anticancer agent followed by examination of each organ. Multiple tumor cell types can also be used to evaluate the efficacy of the tested drug or drug candidate on different types of tumors.

The tool can be utilized for the culturing of cells which require exogenous factors from other cell types without physically mixing the cell types, as the different cell types are placed in different wells, with the overlaying medium allowing the exchange of metabolites and/or secreted biomolecules.

The details of various embodiments of the inventions are set forth in the accompanying drawings and the description below. Other features and advantages of the invention will be apparent from the description, the drawings, and the claims.

### BRIEF DESCRIPTION OF THE DRAWINGS

FIG. 1A is a perspective view of a conventional multi-well culture plate;

FIG. 1B is a cross-section view of the conventional multi-well culture plate shown in FIG. 1A;

FIG. 2A is a perspective view of a cell culture tool;

FIG. 2B is a cross-section view of the cell culture tool shown in FIG. 2A;

FIG. 3A is a perspective view of another embodiment of a cell culture tool;

FIG. 3B is a cross-section view of the cell culture tool shown in FIG. 3A;

FIG. 4A is a perspective view of a further embodiment of a cell culture tool;

FIG. 4B is a cross-section view of the cell culture tool shown in FIG. 4A;

FIG. 5A is a perspective view of a cell culture tool with separate chambers and multiple wells per chamber;

FIG. 5B is a cross-section view of the cell culture tool shown in FIG. 5A;

FIG. 6 shows part of an organ system of an animal;

FIG. 7 is a flow diagram of evaluating metabolism of an exogenous substance by multiple cell types;

FIG. 8 is a flow diagram of evaluating the toxicity of an exogenous substance on multiple cell types;

FIG. 9 is a flow diagram of establishing a therapeutic index of a drug; and

FIG. 10 shows a cell culture tool with an insert tray.

FIG. 11 is a schematic of a multi-well culture plate (96-well plate) with HCV-infected hepatocytes of the multiple HCV genotypes, and the evaluation of multiple potential anti-HCV compounds at multiple concentrations.

FIG. 12 is a flow diagram of an overall process 200 for screening of anti-HCV agents for preventing the infection by HCV.

FIG. 13 is a flow diagram illustrating a method 300 of screening of anti-HCV agents for prevention of HCV infection using cell co-culture.

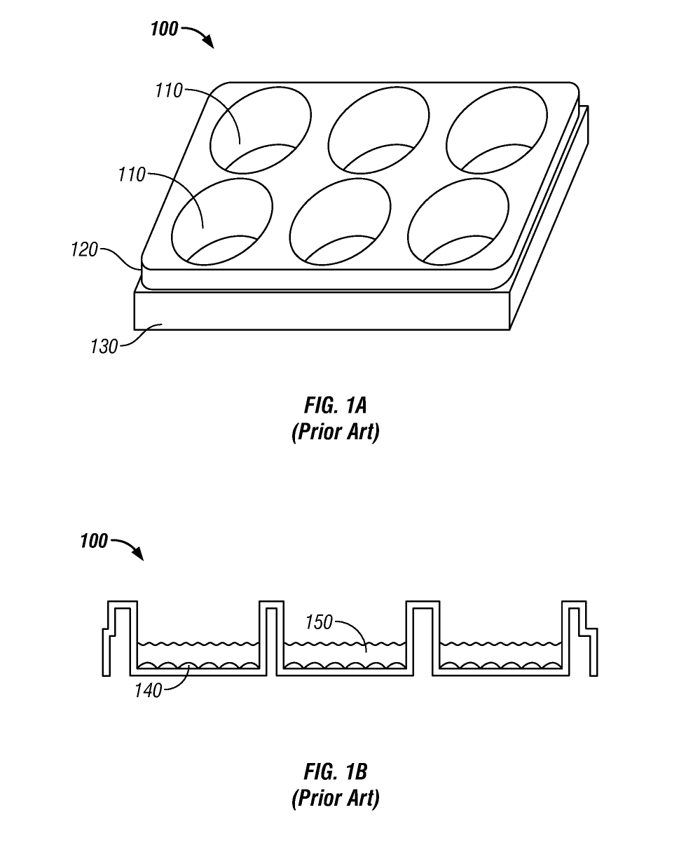

Reference numerals in the drawings correspond to numbers in the Detailed Description for ease of reference.

### DETAILED DESCRIPTION

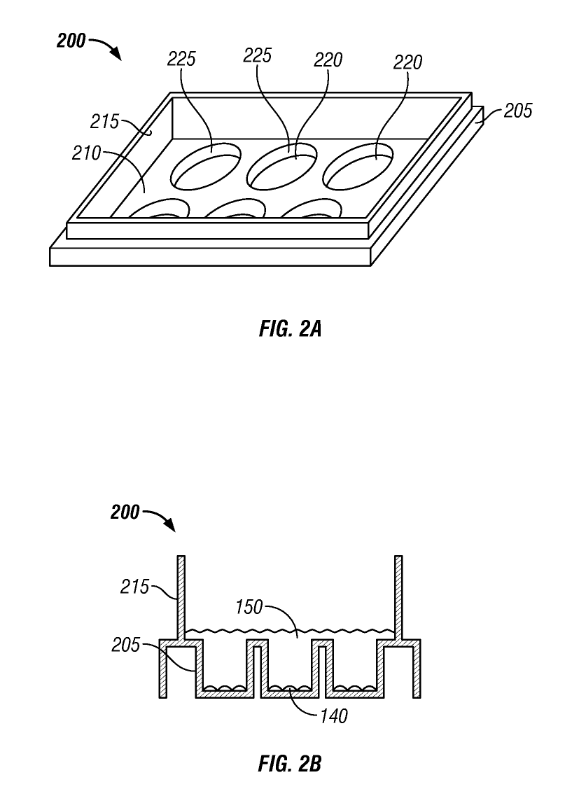

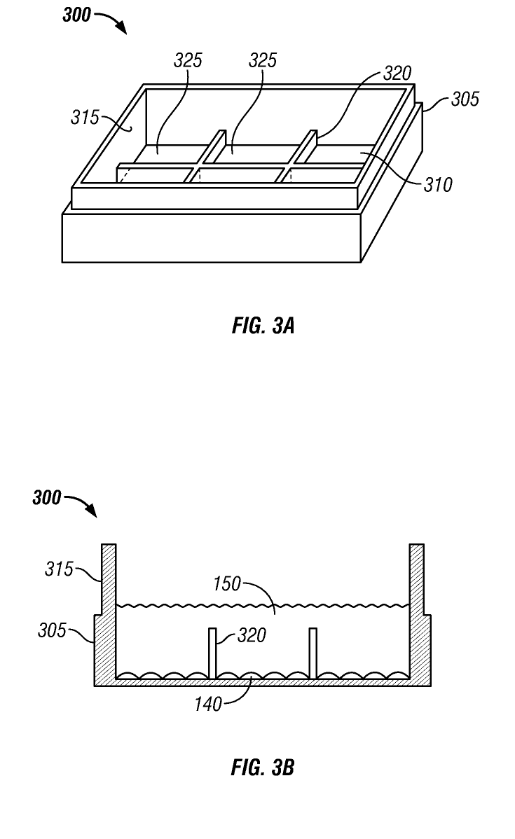

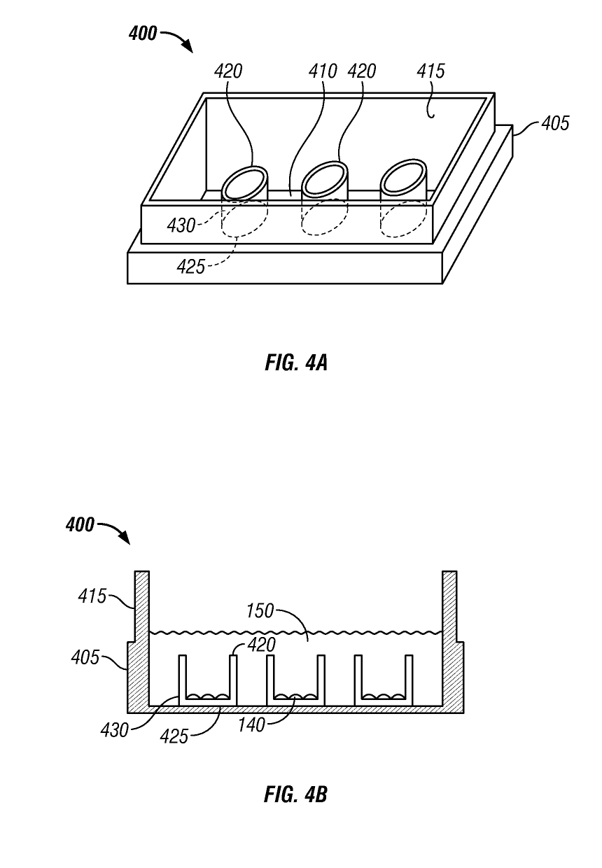

Embodiments of the tool 200, 300, 400 embodying the current invention are shown in FIGS. 2A-5B and FIG. 10. The tool 200, 300, 400 includes multiple wells within each a type of cells can be cultured, but each well can be overfilled or flooded, so that the cells in the different wells can share a common medium. This is achieved by configuring each well as an indentation inside a larger plate (FIGS. 2A and 2B), placing short partitions inside a larger plate (FIGS. 3A and 3B), or placing small inserts inside a larger plate (FIGS. 4A and 4B). However, this invention can be applied to any multi-well format with any number of wells per plate.

Referring to FIGS. 2A and 2B, a multi-well tool 200 of the present invention comprises a body 205 having a substantially planar top surface 210, and an outer wall 215 extending from the body 205. Six wells 220 are formed in the body 205 by depressions in the top surface 210. Each well 220 has a containing wall 225 that may slant downward from or be perpendicular to the flat surface 210.

The overall dimensions of the tool 200 may be about 12.60 cm long and 8.40 cm wide. The body 205 may have a height of 0.20 cm, with the outer wall 215 extending upward from the flat surface 210 approximately 0.15 cm. The height of each containing wall 225 may be 0.05 cm. The wells 220 are configured in a regular array and are separated by approximately 0.02 cm. In another implementation (not shown), the wells are equi-distant from each other by positioning the wells around a circumference of a circle. The dimensions of the tool 200 are merely illustrative, however, the tool 200 is configured to allow overfilling of each well 220 in order to interconnect the wells 220 in a common fluid media while preventing the cells in the individual wells 220 from drowning.

Referring to FIGS. 3A and 3B, a multi-well tool 300 includes a body 305 having a planar top surface 310, surrounded by an outer wall 315. Partitions 320 are positioned on the top surface 310 to divide the space bounded by the outer wall 315 into six wells 325. The outer wall 315 extends upward 0.15 cm from the top surface 310 and the height of the partitions is approximately 0.05 cm. Thus, each well 325 can be overfilled to interconnect the wells 325 in a fluid medium.

The partitions 320 may be bonded to the top surface 310 and the outer wall 315. In another implementation, the partitions 320 may be removable.

Referring to FIGS. 4A and 4B, a multi-well tool 400 includes a body 405 having a planar top surface 410, surrounded by an outer wall 415. Inserts 420 are placed on the flat surface 410, with each insert defined by a bottom 425 and a containing wall 430. The height of the containing wall is about 0.05 cm and the height of the outer wall extends 0.15 cm from the top surface 410. In other implementations, the inserts 420 may comprise cups, dishes, or a tray that may be removed from the top surface 310.

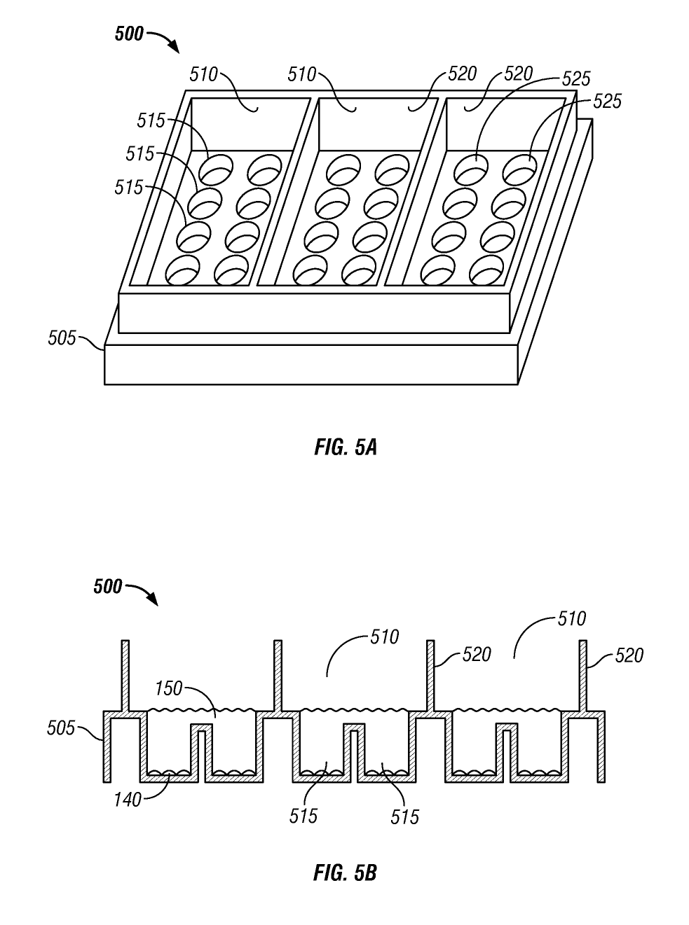

The multi-well plates as described in FIGS. 2A-4B can be grouped to form a cell culture tray 500 as a single body 505 with multiple compartments or chambers 510 (FIGS. 5A and 5B), each compartment 510 having multiple wells 515, to allow experimentation with different cell selections, liquid medium, or a different exogenous substance in each compartment. Limiting walls 520 surrounding each compartment 510 are higher than the containing walls 525 of the individual wells 515 within that compartment 510, with the limiting walls 520 having a height of 0.20 cm and each well 515 inside the larger body 505 having a height of 0.04 cm.

The tool 200-500 may be formed of various suitable materials. In one implementation, the tool 200-500 is formed of a substantially rigid, water-insoluble, fluid-impervious, typically thermoplastic material substantially chemically non-reactive with the fluids to be employed in the assays to be carried out with the tool 200-500. The term “substantially rigid” as used herein is intended to mean that the material will resist deformation or warping under a light mechanical or thermal load, which deformation would prevent maintenance of the substantially planar surface, although the material may be somewhat elastic. Suitable materials include, for example, polystyrene or polyvinyl chloride with or without copolymers, polyethylenes, polystyrenes, polystyrene-acrylonitrile, polypropylene, polyvinylidine chloride, and the like. Polystyrene is a material that can be used as it is the common polymer used for cell culture vessels, inasmuch as it characterized by very low, non-specific protein binding, making it suitable for use with samples, such as, for example, blood, viruses and bacteria, incorporating one or more proteins of interest. Glass is also a suitable material, being used routinely in cell culture vessels and can be washed and sterilized after each use.

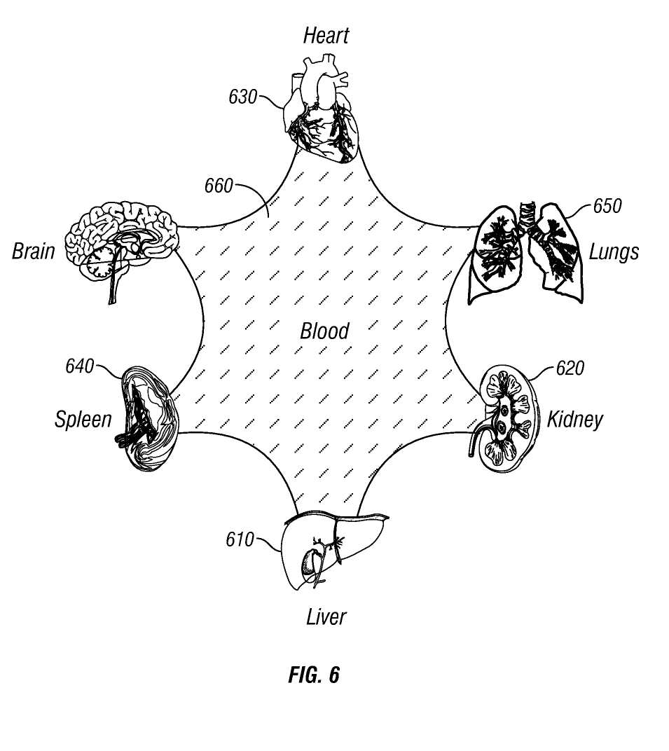

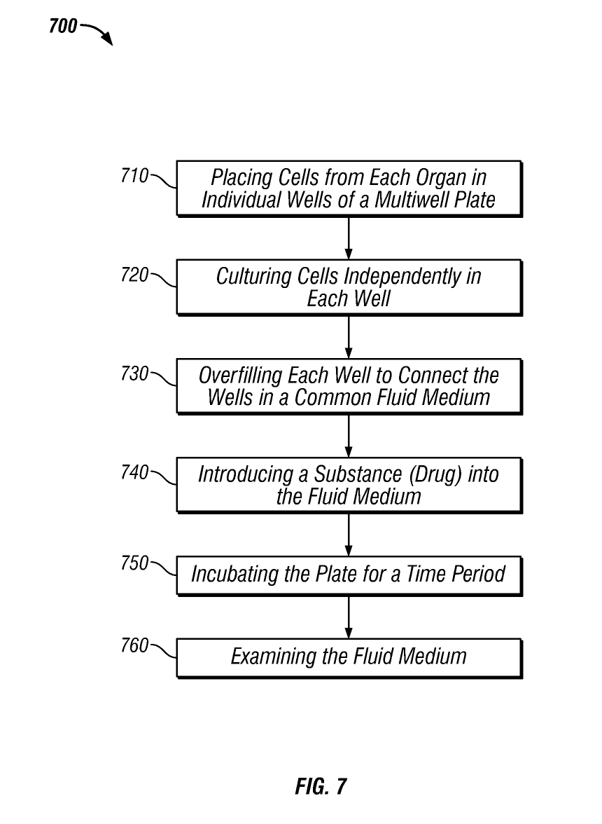

The cell culture tool can be used to test drug metabolism. As shown in FIG. 6, the major organs that are known to metabolize drugs are the liver 610, intestines and kidneys 620, whereas other organs such as the heart 630, spleen 640, lungs 650, and blood vessels 660 also possess specific metabolizing pathways. Referring to FIG. 7, method of using the cell culture tool includes evaluating metabolism of an exogenous substance by multiple cell types 700. Using the tool, the cells from major organs including the liver, intestines, kidneys, heart, spleen, lungs, and brain are placed in the multiple well plate, with cells from each organ placed separately in individual wells (operation 710). For instance, in the six-well format, liver cells are placed in well 1, intestines in well 2, kidneys in well 3, heart in well 4, spleen in well 5 and lungs in well 6. Each cell type can be cultured (operation 720) using different attachment substrate and culture medium, for instance, liver cells are best cultured on collagen and require supplementation with insulin and dexamethasone, spleen cells are cultured in agar suspension, etc. After each cell type is established, the plate can be “flooded” by overfilling each well (operation 730), with the cells from the different wells sharing a common liquid medium. The exogenous substance, such as, for example, a drug, a drug candidate, an environmental pollutant, or a natural product, can be added to the medium (operation 740) and incubated for specific time periods (operation 750). After incubation, the medium can be collected for the examination of the extent of metabolism (how much of the parent substance is remaining), or metabolic fate (what are the identities of the metabolites), using established analytical methods (operation 760).

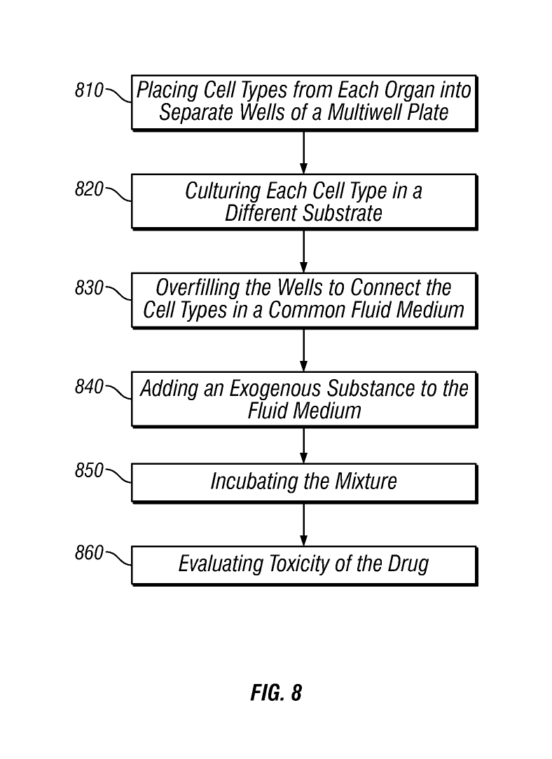

Referring to FIG. 8, another method 800 of using the cell culture tool includes evaluating the toxicity of an exogenous substance on multiple cell types. The major organs that are susceptible to drug toxicity are the liver, intestines, kidneys, heart, spleen, lungs, and brain. Using the tool, the cells from the liver, intestines, kidneys, heart, spleen, lungs, brains and blood vessels, are placed in the multiple well plate (operation 810). Cells from each organ are placed in individual wells. For instance, in an eight-well format, liver cells are placed in well 1, intestines in well 2, kidneys in well 3, heart in well 4, spleen in well 5, lungs in well 6, brain in well 7, and blood vessels in well 8. Each cell type can be cultured using a different attachment substrate and culture medium (operation 820), for instance, liver cells are best cultured on collagen and require supplementation with insulin and dexamethasone, spleen cells are cultured in agar suspension, etc. After each cell type is established, the plate can be “flooded” by overfilling each well, with the cells from the different wells sharing a common liquid medium (operation 830). The exogenous substance, such as, for example, a drug, a drug candidate, an environmental pollutant, or a natural product, is added to the medium (operation 840). The mixture is then incubated for specific time periods (operation 850). After incubation, the medium can be removed, and each individual cell type can be evaluated for toxicity (operation 860) morphologically, such as, for example, microscopic analysis, and by a biochemical analysis, such as, for example, lysed with detergent for the measurement of ATP content of the cells in each individual well.

The cell culture tool can also be used to evaluate drug efficacy and safety. In drug discovery, intact cells are used as indicators of drug efficacy. For instance, liver cells are used to evaluate the effect of a drug on cholesterol synthesis in order to develop a novel inhibitor of cholesterol synthesis as a drug to lower the cholesterol level in patients with high levels of cholesterol. A culture can be applied with cells from multiple organs as described above to evaluate the effects of a drug candidate on cholesterol synthesis in multiple organs. The method can be used to evaluate efficacy, metabolism and toxicity simultaneously using the culture system.

For instance, a “therapeutic index” of a potential new drug to treat high cholesterol levels can be evaluated by using liver cells as indicator cells to determine the effectiveness and toxicity of the drug. Efficacy can be measured in the presence of metabolism of all key cell types, thereby mimicking an in vivo situation where metabolism may lower the efficacy (or increase the efficacy) of the new drug.

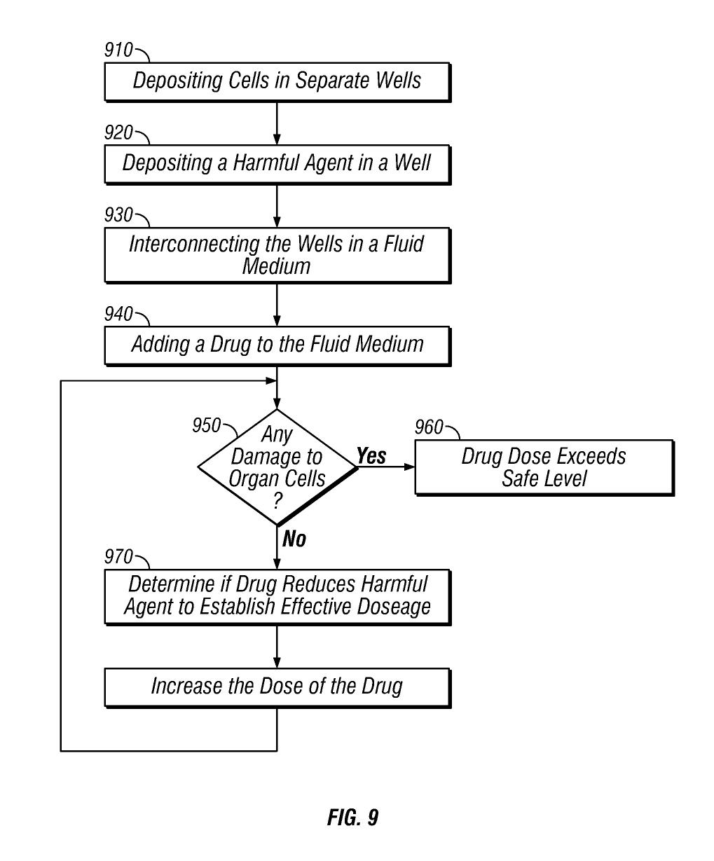

Referring to FIG. 9, a method 900 of establishing a therapeutic index of a drug includes depositing cells in separate wells of the multi-well plate (operation 910), depositing a harmful agent, such as, for example, tumor cells, in another of the wells (operation 920), interconnecting the wells with a fluid medium (operation 930), and adding a drug to the fluid medium (operation 940).

Safety is evaluated by determining the effect of the drug on the various organ cells (operation 950). If the drug damages any of the organ cells, the drug doseage is deemed to exceed a safe level (operation 960). If the healthy cells are intact, the effect of the drug to reduce the harmful agent is examined. If the harmful agent is reduced, the result is recorded as an effective dose level (operation 970). The dose of the drug is then increased (operation 980) and the process is repeated.

The tool also may be used in a high throughput screening (HTS) process to allow evaluation of a large number of potential drug candidates. In this method, a robotic system is utilized with multi-well plates to perform experimentation. By using a multi-compartment tool as described herein, HTS with co-cultured multiple cell types can be performed for efficacy, toxicity, and metabolism as described above.

Still a further method includes evaluation of co-culture conditions. Some cell types can enhance the culturing of an otherwise difficult to culture cell type. This is routinely performed by trial and error. Using the HTS format, the effects of different cell types on the growth of a difficult to culture cell can be examined. For instance, to evaluate which cells are best to maintain the differentiation of cultured liver cells, liver cells can be co-cultured with cell type 1 (e.g. endothelial cells) in compartment 1; cell type 2 (e.g. 3T3 cells) in compartment 2, and so on. At the end of co-culturing, the properties of the liver cells can be evaluated without complications by the co-cultured cells.

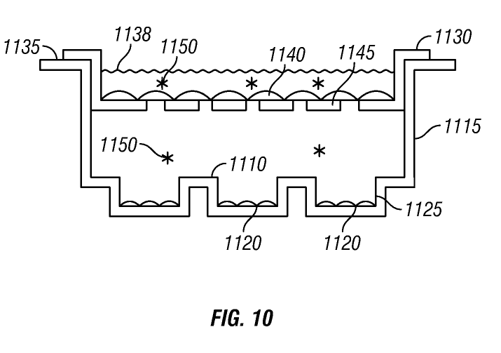

Referring to FIG. 10, a cell culture tool 1000 is shown with an adaptation to measure drug absorption. The cell culture tool 1000 comprises a body 1105 having a substantially planar top surface 1110 surrounded by an outer wall 1115. Six wells 1120 are formed in the body 1105 by depressions in the top surface 1110. Each well 1120 has a containing wall 1125 that is perpendicular to the flat surface 1110.

An insert tray 1130 rests on a lip 1135 at the top of the outer wall 1115. The insert tray 1130 includes a chamber 1138 with a porous membrane 1145 that is positioned inside the outer wall 1115.

Intestinal cells 1140 are placed at the bottom of the chamber 1138 proximate to the membrane. When the tool 1000 is filled, the fluid level rises through the membrane 1140 and a drug 1150 is added to the chamber 1138. The drug 1150 is “absorbed” when it permeates the membrane 1140 to interact with the cells 1120. Thus, the amount of absorption can be measured to simulate absorption of the drug within the intestines.

The inventor has developed an improved method for screening of anti-HCV drugs that may have efficacy for preventing the hepatitis C virus infection. The method involves the isolation and cryopreservation of HCV infected hepatocytes from multiple infected individuals to compile a collection, or bank, of hepatocytes that represents the various HCV genotypes. The isolated and cryopreserved hepatocytes are stored in a cryopreservation bank that represents the various genotypes of the hepatitis C virus. These stored hepatocytes (HCV donor cells) then can be cultured in a culture medium along with uninfected hepatocytes (HCV recipient cells), exposed to anti-HCV agents in the presence of the uninfected hepatocytes, and screened for HCV RNA or protein production. At various times after incubation with the test articles, i.e., the anti-HCV agents, HCV content of the cultures are quantified by quantification of HCV RNA or HCV proteins. An effective anti-HCV agent will lead to a prevention of HCV infection of uninfected hepatocytes by the HCV infected hepatocytes. Using the cryopreservation bank of the genotypes of the HCV and multi-well culture plates, there is now the ability to simultaneously screen in parallel multiple anti-HCV agents against multiple HCV genotypes.

In humans the liver cells (hepatocytes) are the cells where HCV replication occurs. Therefore, human hepatocytes in a culture represent a physiologically relevant model for the evaluation of anti-HCV agents, thereby providing an in vitro model that corresponds to the in vivo condition. To obtain the hepatocytes, the cells are isolated from liver tissue and then preserved using cryopreservation. Thus, one of the aspects of the invention is the isolation and cryopreservation of hepatocytes obtained from the livers of HCV-infected patients.

One of the other aspects of the invention is the collection of hepatocytes from various HCV patients in numbers of patients sufficient to represent the various HCV genotypes. These hepatocytes are obtained from the liver tissue of the HCV infected patients and processed to isolate the cells from the liver tissue, preservation solution, blood, and the like.

The hepatocytes are stored in a cryopreservation bank and the cryopreserved cells later can be thawed and cultured for the production of the hepatitis C virus. One of the other aspects of the invention, therefore, is the culturing of the cryopreserved cells for replication as well as multiplication and therefore the production of the hepatitis C virus. Thus, the invention relates to the use of hepatocytes that are infected by HCV, and are capable of sustained production of the hepatitis C virus.

The invention is based in one aspect on the use of a novel cell culture apparatus (see U.S. Pat. No. 7,186,548 B2, the contents of which are incorporated herein in their entirety by reference) for the evaluation of HCV infection. The apparatus is a co-culture tool which allows the culturing of different cell types as physically separated cultures, but interconnected by an overlying medium. The culture apparatus is called the Integrated Discrete Multiple Organ Co-culture system (IdMOC™). Thus, one of the aspects of the invention is the co-culturing of HCV-infected hepatocytes (HCV donor cells) and uninfected hepatocytes (HCV recipient cells) in the IdMOC™.

One of the other aspects of the invention is the monitoring of replicating HCV in the HCV recipient cells. Yet another aspect of the invention is to provide a novel screening process for agents that can prevent HCV infection. The invention also relates to the use of hepatocytes that are infected by HCV, and are capable of sustained production of the hepatitis C virus (HCV).

The expression “replication of the HCV” designates the molecular process or processes leading to the synthesis of a strand of negative polarity which will serve to engender new strands of positive polarity constituting the genomic material of the HCV.

The expression “production of the HCV” describes the possibility for a given cell to reproduce infectious particles of the hepatitis C virus (viral multiplication cycle).

The expression “in a suitable culture medium” describes the medium in which the cell line is best able to grow. The culture medium can be, for example, the DMEM/F12 medium with 10% FCS (fetal calf serum) medium supplemented by the elements necessary for the differentiated properties of human hepatocytes, particularly insulin, dexamethasone, selenium, and transferring.

The expression “RT PCR” designates real time polymerase chain reaction used for amplification of a piece of RNA across several orders of magnitude, generating million or more copies of a particular RNA sequence.

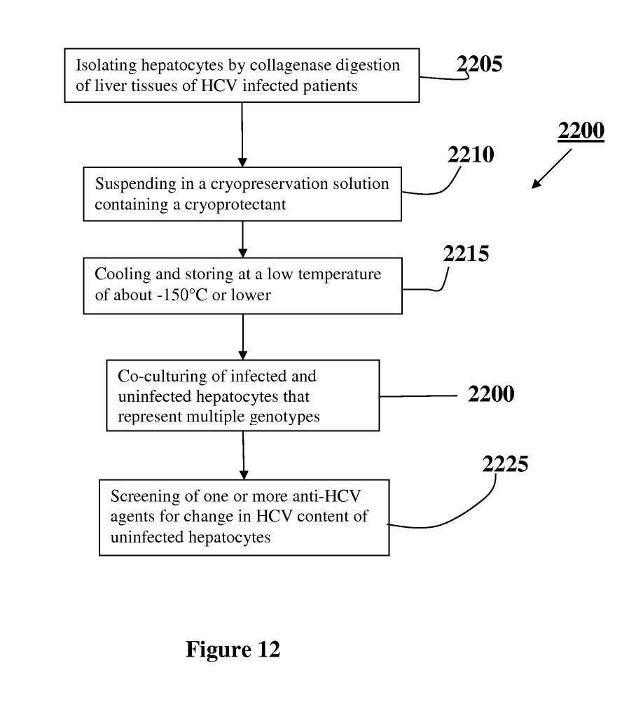

FIG. 12 illustrates the overall process 2200 for screening of anti-HCV agents. In a first step 2205, hepatocytes are isolated by collagenase digestion from livers obtained from patients infected with hepatitis C virus as well as from livers of uninfected individuals. The hepatocytes can be used immediately or cryopreserved for use at a later time.

In a second step 2210, hepatocytes from HCV-infected and uninfected livers are co-cultured in the IdMOC™. For instance, using an IdMOC™ with 6 inner wells within each containing well, three wells can be used for the culturing of the infected hepatocytes (donor hepatocyte cells), and three for the uninfected (recipient hepatocyte cells) hepatocytes. The isolated hepatocytes are then suspended in a suitable cryopreservation solution with cryoprotectant, such as DMEM/F12 medium with 10% fetal calf serum and 10% dimethyl sulfoxide.

In a third step 2215, the cells are cooled and stored in the cryopreservation solution at a suitable temperature, such as approximately −150° C. or lower.

To prepare the cells for screening anti-HCV agents for preventing HCV infection, the cells are co-cultured for hepatitis C replication (step 2200). In this step, the cells are thawed and co-cultured on a suitable substratum, particularly a collagen-coated plate with multiple wells. The process of screening one or more anti-HCV agents (step 2225) includes introduction of the various anti-HCV compounds into the co-culture medium containing the cultured hepatocytes representing multiple genotypes of HCV and uninfected hepatocytes, extraction of the total RNA of the cells, and analysis for synthesis of RNA of HCV in uninfected hepatocytes. Effective anti-HCV agents will prevent the increase of HCV content in the uninfected hepatocytes.

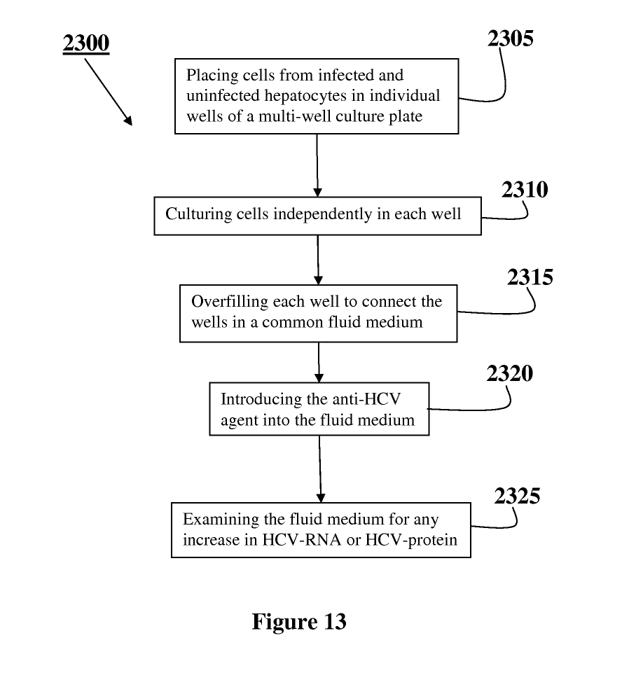

Referring to FIG. 13, a method 2300 to evaluate a level of HCV infection through quantification of HCV production by infected hepatocytes using a co-culture of infected and uninfected hepatocytes includes a first step 2305, in which the HCV infected and uninfected hepatocytes are placed in individual wells of a multiwell culture plate.

In a second step 2310, using the cell culture tool, the infected and uninfected hepatocytes are cultured independently in each well. For example, in the six well format, three wells can be used to culture uninfected hepatocytes and the remaining three wells can be used to culture infected hepatocytes.

In a third step 2315, the wells are connected to each other by over filling each well with a common fluid medium.

In a fourth step 2320, the anti-HCV agent is added to the connecting fluid medium. Finally in step 2325, the fluid medium is examined for any increase in HCV RNA or HCV protein to evaluate the level of HCV production in uninfected hepatocytes. The invention also relates to the cells obtained by implementing the processes as defined and described above.

### EXPERIMENTAL PROCEDURES

### Isolation, Cryopreservation, and Culturing of HCV-Infected Human Hepatocytes:

1. Human hepatocytes are isolated from livers of hepatitis C-infected patients by perfusion. The livers are obtained from organ procurement organizations (e.g. NDRI; IIAM).

2. The livers are first perfused with an isotonic solution (e.g. Hanks Balanced Salt Solution) to remove blood and organ-preservation solutions, followed by perfusion with an isotonic solution containing a suitable concentration of collagenase (e.g. 0.5 mg/mL). The collagenase treatment digests the liver tissue and releases highly viable hepatocytes.

3. The hepatocytes are washed by low speed centrifugation (e.g. 50×g) in an isotonic solution, and resuspended in a solution containing cryoprotectants (in particular, DMEM/F12 medium supplemented with 10% fetal calf serum and 10% dimethylsulfoxide). The collected hepatocytes can be further purified by density gradient centrifugation prior to resuspension. The density gradient may be 30% by volume of Percoll®. The centrifugation may be 100×g.

4. The cells are cryopreserved in a programmable freezer at a constant rate of freezing, particularly −1° C. per minute until a suitable low temperature, particularly −70° C. or lower, is reached.

5. The cryopreserved cells are stored at a suitable temperature, particularly about −150° C. or lower, using a suitable apparatus, particularly a liquid nitrogen cryogenic storage system.

### Development of a Cryopreserved Hepatocyte Bank:

The cryopreserved cells obtained above are collected together to form a cryopreserved hepatocyte bank. The hepatitis C virus is presently classified into six genotypes, with several subtypes within each genotype. The subtypes are broken down into quasispecies based on their genetic diversity. A collection (“bank”) of hepatocytes from multiple HCV patients provides a complete set of the multiple genotypes of HCV to allow studying the biology of the various genotypes and development of potential measures to inhibit the spread of the infection by the individual genotypes.

### Cell Maintenance Conditions for HCV-Infected Human Hepatocytes:

1. Hepatocytes of multiple genotypes are retrieved from cryopreservation and thawed in a 37° C. water bath.

2. The cells are suspended in DMEM/F12 medium.

3. The suspended HCV donor hepatocytes are co-cultured with HCV recipient hepatocytes in IdMOC™. For instance, using an IdMOC™ with 6 inner wells within each cell plate, three wells can be used for the culturing of the donor hepatocyte cells, and three for the recipient hepatocyte cells.

4. After the cells are attached, the cells are overlaid with Matrigel® by changing the medium to that containing 0.25 mg Matrigel®. The co-culture medium is placed daily.

In step 3 above, the cells can be cultured in a variety of cell culture tools as are known in the art.

In another implementation, each vessel comprises a cup connected to the body and each cup has a top edge below the rim of the outer wall.

### Evaluation of HCV Production

Quantification of HCV production is important for the development of anti-HCV screens (see below) for the prevention of HCV infection. This evaluation includes the following steps:

1. Samplings of cells.

2. Extracting the RNA of these cells.

3. Quantifying the RNA of the HCV either by RT-PCR, or by hybridization of the RNAs on filters.

4. If the viral infection does not lead to a lysis of the cells, the multiplication of the HCV can be observed by indirect immunofluorescence using antibodies directed against proteins of the HCV.

### Process for Screening Anti-HCV Agents:

1. HCV-infected hepatocytes together with uninfected hepatocyte are subjected in parallel to the action of multiple, potential anti-HCV agents or compounds (test articles) at a range of concentrations.

2. After incubation with the test articles, HCV content of the cultures are quantified by quantification of HCV RNA or HCV proteins

3. Hepatocytes with different genotypes, may be used as anti-viral measures, and may be genotype-specific.

4. Effective anti-HCV agents will prevent the increase of HCV content in the uninfected hepatocytes.

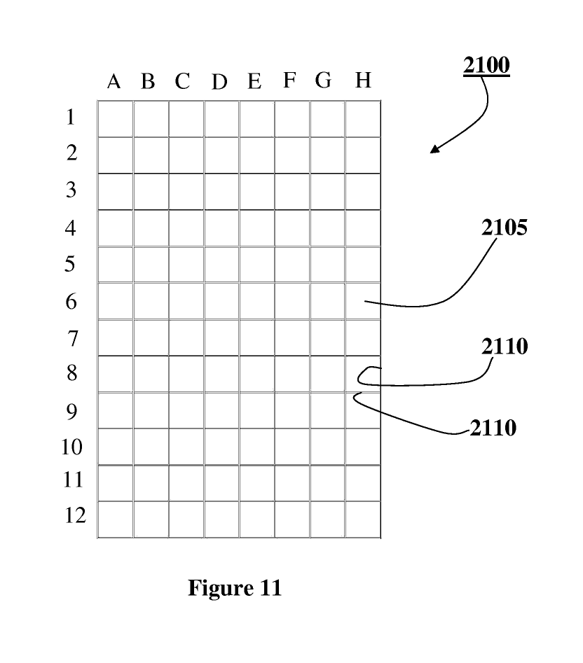

Referring to FIG. 11, the screening can be performed in a multi-well culture plate 100 (e.g., a 96-well plate) by using HCV-infected hepatocytes of the multiple HCV genotypes. The multi-well plate 2100 includes wells 2105 that are defined by walls 2110. The columns A-H are used to test different anti-HCV agents for treating HCV. Thus, agent 1 is placed in each well of column A, agent 2 is placed in each well of column B, etc. The rows 1-12 are used to provide HCV infected cells representing the different genotypes and subtypes. For example, row 1 may be used for subtype 1a, row 2 may be used for subtype 1b, row 3 may be used for subtype 2a, row 4 may be used for subtype 3a, row 5 may be used for subtype 4a, etc. In this manner, multiple agents for treating HCV may be simultaneously tested on multiple genotypes and subtypes of HCV infections. This ability to simultaneously test multiple agents against multiple genotypes will increase the speed and efficiency by which agents can be tested to treat HCV and thereby improve the likelihood that suitable treatment agents will be found quicker.

While several particular forms of the invention have been illustrated and described, it will be apparent that various modifications and combinations of the invention detailed in the text and drawings can be made without departing from the spirit and scope of the invention. For example, references to materials of construction, methods of construction, specific dimensions, shapes, utilities or applications are also not intended to be limiting in any manner and other materials and dimensions could be substituted and remain within the spirit and scope of the invention. Accordingly, it is not intended that the invention be limited, except as by the appended claims.

What is claimed is:

1. A method for co-culturing HCV-infected and uninfected human hepatocytes to screen for agents that prevent the transmission of HCV, the method comprising
retrieving HCV infected and uninfected hepatocyte cells from a cryopreservation bank;
thawing the cells in a warm water bath;
suspending the cells in a medium;
culturing the cells in a plate with multiple inner wells;
interconnecting the inner wells with a fluid medium;
providing one or more anti HCV agents; and
quantifying the HCV content of the co-culture by quantification of HCV RNA.

2. The method of claim 1, wherein the anti-HCV agent is added to the fluid medium used for interconnecting the wells.

3. The method of claim 1, wherein multiple anti-HCV agents are used.

4. The method of claim 1, wherein the multiple anti-HCV agents are added at different concentrations.

5. The method of claim 1, wherein the co-cultured uninfected hepatocytes and infected hepatocytes are connected by a fluid medium.

6. The method of claim 5, wherein the fluid medium used for interconnecting the wells is DMEM/F12 medium containing 10% of fetal calf serum (FCS), insulin (10 ug/mL), and dexamethasone (100 nM).

7. The method of claim 1, wherein the quantification of HCV RNA is performed by RT-PCR.

8. The method of claim 1, wherein the temperature of the warm water bath is approximately 37° C.

9. The method of claim 1, wherein the suspending medium is DMEM/F12 medium containing 10% of fetal calf serum (FCS), insulin (10 ug/mL), and dexamethasone (100 nM).

10. The method of claim 1, wherein the cell plate comprises a collagen-coated plate.

11. The method of claim 1, wherein the cell plate has six inner wells.

12. The method of claim 11, wherein three wells are used for culturing infected hepatocytes.

13. The method of claim 11, wherein three wells are used for culturing uninfected hepatocytes.

14. The method of claim 1, different genotypes of HCV infected cells are co-cultured with uninfected hepatocytes in multiple cell plates.

15. A method for co-culturing infected and uninfected hepatocytes in a medium to evaluate a level of HCV infection through quantification of HCV production by infected hepatocytes, the method comprising:
extraction of total RNA of uninfected hepatocytes;
quantification of RNA of uninfected hepatocytes; and
quantification of an HCV titer of the medium and infected hepatocytes by quantification of RNA of HCV.

16. The method of claim 15, wherein the quantification of RNA of HCV is performed by RT-PCR.
